(19) United States
(12) Patent Application Publication
(10) Pub. No.: US 2004/0067181 A1
(43) Pub. Date: Apr. 8, 2004

(54) Ultrasonic fragrance generating device

(76) Inventors: Zong-Fu Shin, Taipei (TW); Hsiao-Li Chang, Taipei (TW)

Correspondence Address: Zong-Fu Shin, PO Box 82-144, Taipei, TW

(21) Appl. No.: 10/265,240

(22) Filed: Oct. 7, 2002

(51) Int. Cl. A62B 7/08; A61L 9/00

(52) U.S. Cl. 422/305; 422/306; 422/307; 422/123

(57) ABSTRACT

An ultrasonic fragrance-generating device is disclosed and the device comprises an inner cup, a housing a covering ring, an oscillating unit and a heat dissipation fan and the mouth of the housing being inserted into the inner edge of the mouth of the inner cup and the mouth of the inner cup being covered by the covering ring and the cup edge of the housing being covered by a hood and the housing being an inner layered housing and an external layered housing, and the inner layer housing being a hole at the bottom section thereof for the mounting of the oscillation unit and the bottom face of the housing being mounted with the heat dissipation fan, characterized in that the wall of the hood is mounted with at least a layer of magnetic solenoid and the positive pole of the magnetic solenoid faces the inner cup, forming into energy conduit for magnetized fluidization.

### BACKGROUND OF THE INVENTION

1. Field of the Invention

The present invention relates to a novel structure of an ultrasonic fragrance generating device characterized in that the wall of the hood is mounted with at least a layer of magnetic solenoid and the positive pole of the magnetic solenoid faces the inner cup, forming into energy conduit for magnetized fluidization.

2. Description of the Prior Art

Fragrant compounds are well known in the art for masking foul odors with more pleasing scents or odors which are introduced into enclosed areas by the release of natural and/or synthetic compounds. The medical art of aromatherapy administers by inhalation, ingestion or skin absorption small amounts of the essential oils of plants that are believed to possess therapeutic properties. Also, fragrant compounds may be added to the construction materials of products that are used in odiferous environments in an attempt to mask unpleasant odors.

One form of known deodorizing device involves evaporation of a solid or liquid containing a fragrance compound and which is evaporated through the application of heat or the opening of a vent between a chamber within a body containing the fragrant compound and the surrounding enclosed area. Other known prior art examples introduce the fragrant aroma into the surrounding environment by atomization of a liquid fragrance compound or by blowing air past a reservoir of a liquid or solid material containing the fragrant compound.

FIGS. 1A to 1D show the structure of a conventional ultrasonic fragrance generating device comprising an inner cup 10, a housing 20, a covering ring 30 a hood 40, an oscillation unit 50 and a heat dissipation fan 60. The mouth 21 of the housing 20 in inserted at the inner edge of the inner cup mouth 11 which is covered by the covering ring 30. The cup edge of the housing is covered by the hood 40, and the housing 20 can be divided into an inner housing 22 and an outer housing 23 and the bottom section of the inner layer housing 22 is provided with a hole 24 for engaging with the oscillation unit 50 which comprises a waterproof pad 51, a screw securing ring 52, an oscillation device 53, a waterproof O-shaped ring 54, a square shaped oscillation seat 55 and a securing frame 56, and the bottom face of the external layer of the housing 23 is disposed with the heat dissipation fan 60. the air vent 42 at the top end of the tube wall 41 of the hood is inserted with a nozzle 43. The drawbacks of the conventional ultrasonic fragrance generating device are that:

(1) the turbulent formed surround the hood of the fragrance generating device will change to big droplet which losses the efficiency of atomizing,

(2) the components of the fragrance generating device may be damaged as a result of poor heat dissipation,

(3) the fragrance cannot be evenly distributed for the reason that the atomized fragrance has not undergone a mixing treatment, and

(4) the fastening frame is easily broken if the locking pressure of the locking screws is not evenly applied thereto.

Therefore, it is an object of the present invention to provide a novel structure of an ultrasonic fragrance generating device which can obviate and mitigate the above-mentioned drawbacks.

### SUMMARY OF THE PRESENT INVENTION

Accordingly it is an object of the present invention to provide an ultrasonic fragrance-generating device having an inner cup, a housing a covering ring, an oscillating unit and a heat dissipation fan and the mouth of the housing being inserted into the inner edge of the mouth of the inner cup and the mouth of the inner cup being covered by the covering ring and the cup edge of the housing being covered by a hood and the housing being an inner layered housing and an external layered housing, and the inner layer housing being a hole at the bottom section thereof for the mounting of the oscillation unit and the bottom face of the housing being mounted with the heat dissipation fan, characterized in that the wall of the hood is mounted with at least a layer of magnetic solenoid and the positive pole of the magnetic solenoid faces the inner cup, forming into energy conduit for magnetized fluidization.

Yet another object of the present invention is to provide an ultrasonic fragrance generating device, wherein a temperature sensing element is disposed at the bottom side of the housing such that when the working temperature increases to 55 degree C., the oscillator will stop its action.

Yet a further object of the present invention is to provide an ultrasonic fragrance generating device, wherein the energy conduit causes the magnetized fluidization and accelerates absorbing and repelling action.

Another object of the present invention is to provide an ultrasonic fragrance generating device, wherein the oscillation unit comprises a waterproof pad, a screw fastening pad, a first waterproof O-shaped ring, a oscillator, a second waterproof O-shaped ring, a cylindrical shaped oscillation seat, and a circular securing frame, and a plurality of locking points at the securing frame tightly seal the oscillation unit at the bottom section of the hole within the inner layer housing.

It is another object of the present invention to provide an ultrasonic fragrance generating device, wherein the mouth of the covering ring is provided horizontally with crossed-shaped partition strip and the center region of the strip is disposed with a mixing plate.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1A is a perspective view of a conventional ultrasonic fragrance generating device.

FIG. 1B is a perspective exploded view of a conventional ultrasonic fragrance generating device.

FIG. 1C is a sectional view of a conventional ultrasonic fragrance generating device.

FIG. 1D is a sectional view of a conventional ultrasonic fragrance generating device, showing the application thereof.

FIG. 2 is a perspective view of the present ultrasonic fragrance generating device in accordance with the present invention.

FIG. 3 is a perspective exploded view of the present ultrasonic fragrance generating device in accordance with the present invention.

FIG. 4 is a sectional view of the present ultrasonic fragrance generating device in accordance with the present invention.

FIG. 5 is a schematic view showing the suction and repelling of fragrant ions by the energy conduit in accordance with the present invention.

FIG. 6 is an enlarged view of the action of the magnetic force of the energy conduit of FIG. 5 in accordance with the present invention.

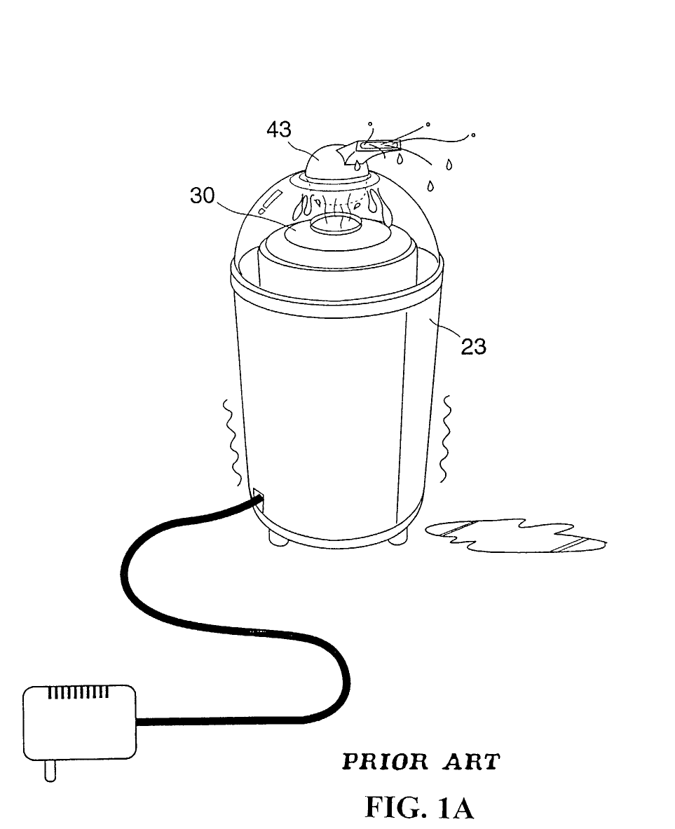

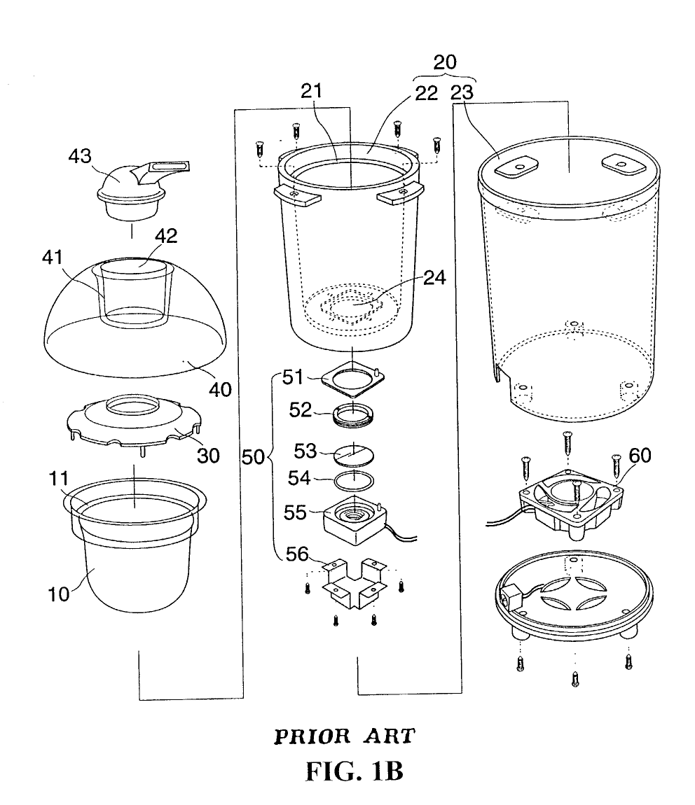

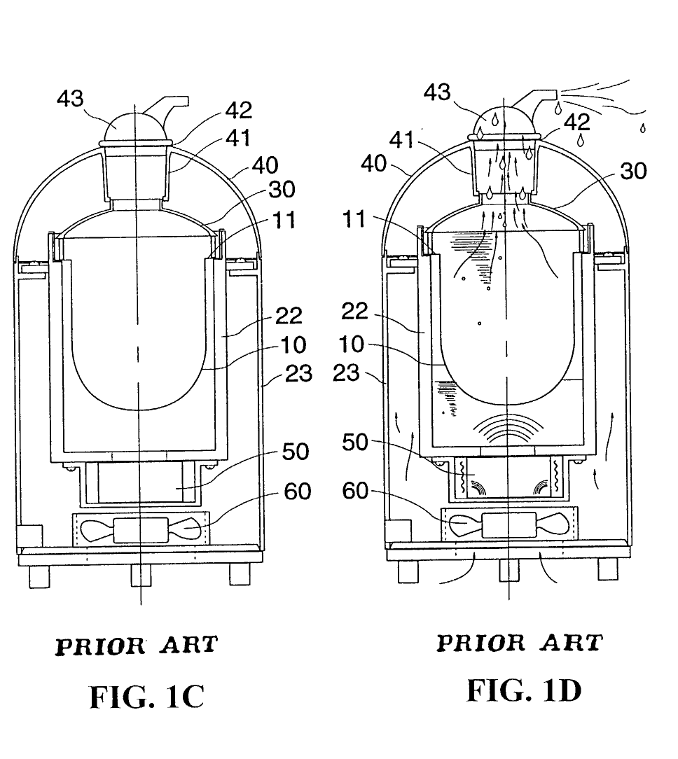

### DETAILED DESCRIPTION OF THE PRESENT INVENTION

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

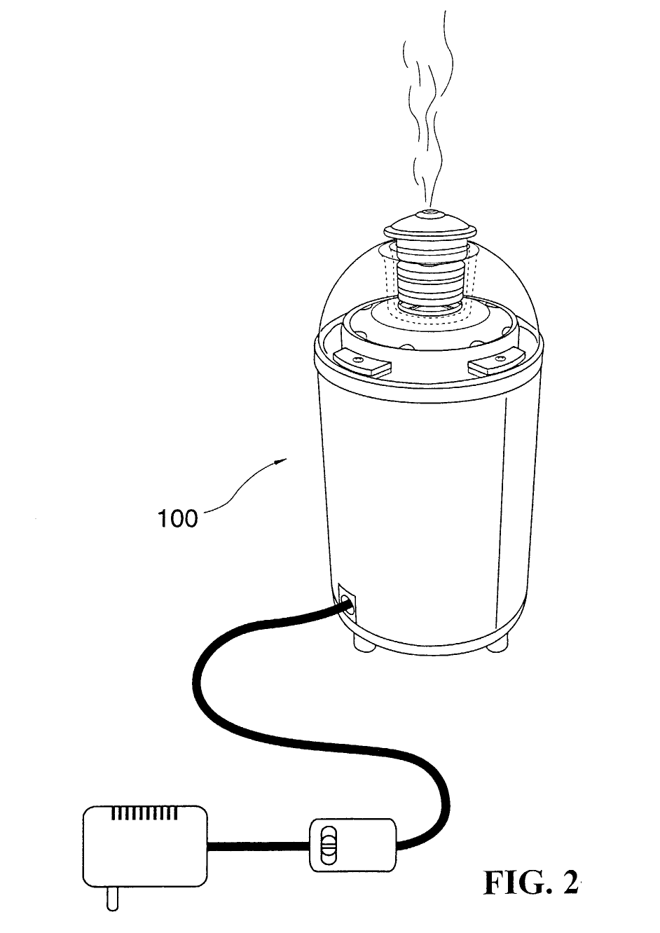

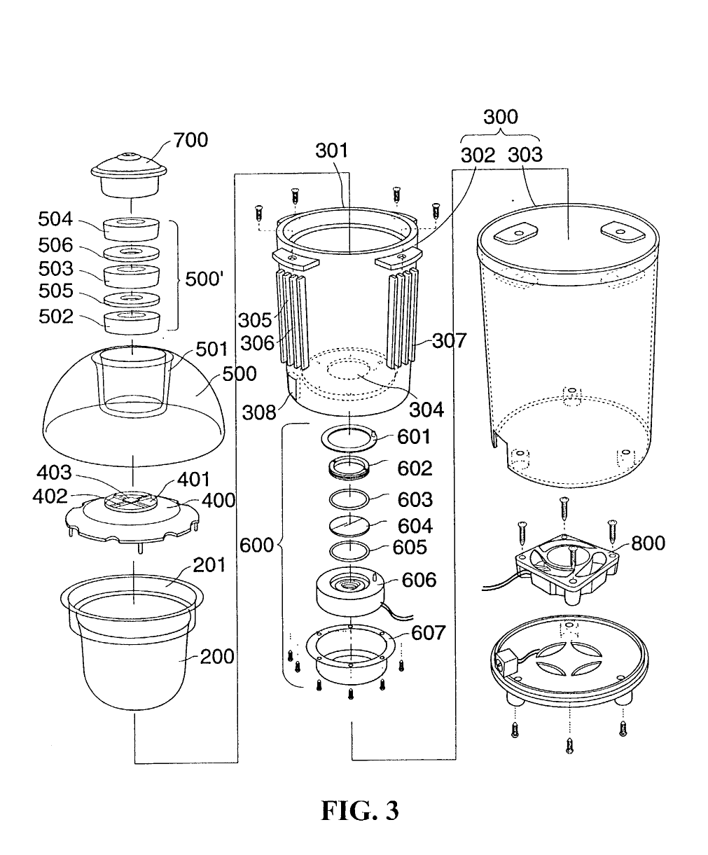

Referring to FIGS. 2 to 4, there is shown the structure of an ultrasonic fragrance generating device 100 comprising an inner cup 200, a cup-shaped housing 300, a covering ring 400 a hood 500, an energy conduit 500′, an oscillation unit 600 and a heat dissipation fan 800. The mouth 301 of the housing 20 is inserted at the inner edge of the inner cup mouth 201 which is covered by the covering ring 400. The cup edge of the housing 300 is covered by the hood 500, and the housing 300 can be divided into an inner housing 302 and an outer housing 303 and the bottom section of the inner layer housing 302 is provided with a hole 304 for engaging with the oscillation unit 600. The inner layer housing 302 is fabricated from good conductive, oscillating material, such as metallic material so as to increase the strength of the amplitude. The bottom face of the outer layer housing 302 is mounted with the heat dissipation fan 800, and the top end opening of the tube wall of the hood 500 is inserted with a nozzle 700. The opening of the nozzle 700 is in communication with the tube wall 501 and are in alignment. In accordance with the present invention, the oscillation unit 600 comprises a waterproof pad 601, a screw fastening pad 602, a first waterproof O-shaped ring 603, a oscillator 604, a second waterproof O-shaped ring 605, a cylindrical shaped oscillation seat 606, and a circular securing frame 607, and a plurality of locking points at the securing frame 607 tightly seal the oscillation unit 600 at the bottom section of the hole 304 within the inner layer housing 302 such that a multiple points sealing is formed. This structure overcomes stress problems as a result of pressure differences due to screw locking to the oscillation device 600. The mouth 401 of the covering ring is provided horizontally with crossed-shaped partition strip 402 and the center region of the strip 402 is disposed with a mixing plate 403. The external side of the inner layer housing 302 is provided with a plurality of heat dissipation plates 305, 306, 307 and the bottom of the housing is mounted with a temperature sensing element 308. When the working temperature reaches 55 degree C., the oscillator stops working, and when the temperature reaches a normal working temperature, the oscillator starts to operate again. This will overcome of damage to the components of the oscillator. The tube wall 501 of the hood 500 is provided with an energy conduit 500′ and is provided at least with a layer of magnetic solenoids 502, 503, 504, and these solenoids 502, 503, 504 will attract the positive pole of the ions to face the inner cup 200. There is a magnetic metallic ring 505, 506 between two layers of the magnetic solenoids 502, 503, 504 and this will form an energy conduit which provides magnetized fluidization.

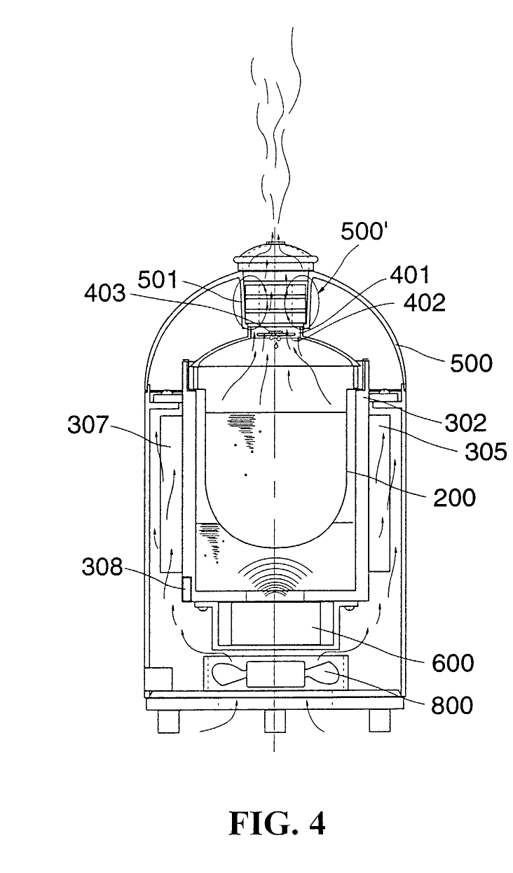

Referring to FIG. 4, a media is inserted into the inner layer housing 302, and the inner cup 200 is placed with fragrance. The oscillation unit 600 and the fan 800 are switched ON. The fluid of the fragrant will be atomized and the heat dispassion plates 305, 307 increase heat dissipation area. The mixing plate 403 mixes the atomized fragrance and then evenly distributed. The mixing plate 403 can be used as a blocking plate to avoid the splashing of water droplets.

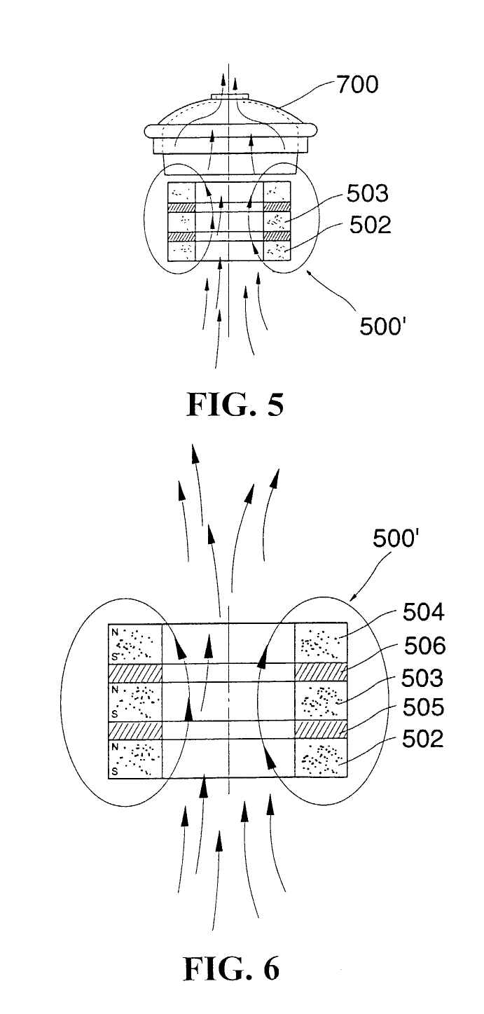

As shown in FIG. 5, the energy conduit causes greater atomized fragrance to repel. As shown in FIG. 6, the atomized fragrance with ions will first to enter into the energy conduit and attracted by the positive pole of the first layer magnetic solenoid to the negative pole of the first layer solenoid 502. Similarly, this happens in the second layer of solenoid 503 and an accelerated force is produced. Therefore, the atomized fragrance is discharged. The magnetic field formed by the magnetic solenoids 502, 503, 504 will surround the surrounding of the conduit wall 501 which directs un-directional ions to move along the positive pole of the magnetic force to the conduit wall 501. The fragrance is thus concentrated and is effectively discharged. The energy conduit will provide efficient dispersion of the atomized fragrance.

It will be envisioned that various substitutions and modifications of the preferred embodiment illustrated in the drawings and described above can be made without departing from the invention as defined in the claims. It will be understood that persons skilled in the art will envision various substitutions of functionally equivalent structure for the structural elements described without departing from the invention.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An ultrasonic fragrance-generating device having an inner cup, a housing a covering ring, an oscillating unit and a heat dissipation fan and the mouth of the housing being inserted into the enter edge of the mouth of the inner cup and the mouth of the inner cup being covered by the covering ring and the cup edge of the housing being covered by a hood and the housing being an inner layered housing and an external layered housing, and the inner layer housing being a hole at the bottom section thereof for the mounting of the oscillation unit and the bottom face of the housing being mounted with the heat dissipation fan, characterized in that the wall of the hood is mounted with at least a layer of magnetic solenoid and the positive pole of the magnetic solenoid faces the inner cup, forming into an energy conduit for magnetized fluidization.

2. The ultrasonic fragrance generating device of claim 1, wherein the energy conduit being engaged with a plurality of layers of magnetic solenoid is provided with a magnetic metallic ring positioned between two magnetic solenoids.

3. The ultrasonic fragrance generating device of claim 1, wherein the oscillation unit comprises a waterproof pad, a screw fastening pad, a first waterproof O-shaped ring, a oscillator, a second waterproof O-shaped ring, a cylindrical shaped oscillation seat, and a circular securing frame, and a plurality of locking points at the securing frame tightly seal the oscillation unit at the bottom section of the hole within the inner layer housing.

4. The ultrasonic fragrance generating device of claim 1, wherein the mouth of the covering ring is provided horizontally with crossed-shaped partition strip and the center region of the strip is disposed with a mixing plate.

5. The ultrasonic fragrance generating device of claim 1, wherein the external side of the inner layer housing is provided with a plurality of heat dissipation plates and the bottom of the housing is mounted with a temperature sensing element.

6. The ultrasonic fragrance generating device of claim 1, wherein the inner layer housing is fabricated from metallic material.
